(19) United States
(12) Patent Application Publication
(10) Pub. No.: US 2019/0313971 A1
(43) Pub. Date: Oct. 17, 2019

(54) NERVE PROBE ATTACHABLE/DETACHABLE TO/FROM SURGICAL INSTRUMENT, AND ENERGY DEVICE SURGICAL INSTRUMENT HAVING NERVE PROBE ATTACHED THERETO

(71) Applicant: PUSAN NATIONAL UNIVERSITY INDUSTRY-UNIVERSITY COOPERATION FOUNDATION, Busan (KR)

(72) Inventor: Byung-Joo LEE, Busan (KR)

(21) Appl. No.: 16/319,339

(22) PCT Filed: Aug. 31, 2016
(86) PCT No.: PCT/KR2016/009737

§ 371 Date: Jan. 19, 2019

(30) Foreign Application Priority Data
Aug. 31, 2016 (KR) 10-2016-0111913
Aug. 31, 2016 (KR) 10-2016-0111923

(51) Int. Cl. A61B 5/00; A61B 5/0492; A61B 18/12

(52) U.S. Cl. CPC A61B 5/4893; A61B 2017/00477; A61B 18/1233; A61B 5/0492

(57) ABSTRACT

The nerve probe attachable and detachable to and from a surgical instrument according to the present invention has a technical feature in that it is detachably mounted to a surgical instrument (a general surgical instrument, an endoscopic surgical instrument, or a robotic surgical instrument) made of an electrically conductive material to allow an electric current to be applied to body tissue by the surgical instrument so that real-time nerve identification and surgery can be performed simultaneously at body tissue coming into close contact with the surgical instrument only by use of the surgical instrument even without suffering from inconvenience of having to frequently replace a separate nerve probe and the surgical instrument, leading to promotion of rapid surgery and improvement of convenience of medical staffs.

### TECHNICAL FIELD

The present invention relates to a nerve probe attachable and detachable to and from a surgical instrument in which it is detachably mounted to a surgical instrument (a general surgical instrument, an endoscopic surgical instrument, or a robotic surgical instrument) made of an electrically conductive material to allow an electric current to be applied to body tissue by the surgical instrument so that real-time nerve identification and surgery can be performed simultaneously at body tissue coming into close contact with the surgical instrument only by use of the surgical instrument even without suffering from inconvenience of having to frequently replace a separate nerve probe and the surgical instrument, leading to promotion of rapid surgery and improvement of convenience of medical staffs, in which the nerve probe can be used by being detachably attached to various forms of the surgical instruments in a simple and easy manner, leading to promotion of acquisition of universality and enhancement of utility, and in which a main body block includes a magnet body or a forceps/a hook/a fitting groove so as to be attached to a rear end of the surgical instrument so that the nerve probe can be used easily without twisting of the wire irrespective of the kind and position of and any changes in posture of the surgical instrument, thus leading to optimization of an environment of surgery that is performed simultaneously with nerve identification, and to a surgical energy device attached with a nerve probe in which an electric wire for probe formed extendedly at a distal end of the surgical instrument to which the nerve probe is detachably mounted so that real-time nerve identification and surgery can be performed simultaneously at body tissue coming into close contact with the surgical instrument only by use of the surgical instrument, leading to promotion of rapid surgery and improvement of convenience of medical staffs, and in which in particular, it is applied to an energy devices for surgical operations, which requires a special attention to be paid to nerve injury due to generation of heat so that nerve identification and early finding of nerve injury caused by heat can be performed in an accurate and smooth manner, leading to minimization of nerve injury during surgery.

### BACKGROUND ART

Nerve injury occurring during a surgical operation is a critical complication in a variety of surgical operations such as otorhinolaryngological surgery, brain surgery, thoracic surgery, spinal surgery, orthopedic surgery, and the like. An intraoperative nerve monitoring system is used to prevent the nerve injury during a surgical operation. In this case, the nerve monitoring system which employs electroneuromyography evaluates the movement of relevant muscles using electromyogram by applying electrical stimulation to a site where a nerve is suspected of being located. To this end, the nerve monitoring system requires a nerve probe that can apply electrical stimulation separately from a surgical instrument.

Nerve injury may occur even though a surgical operation is performed using such a nerve monitoring system. One of causes of occurrence of nerve injury during the surgical operation is that a surgeon does not recognize a nerve located at an abnormal position as a normal nerve and cuts out the nerve. In other words, a conventional nerve monitoring system is a system which applies electrical stimulation to a site where a nerve is suspected of being located using a separate nerve probe to identify the nerve. Thus, in the case where nerve identification is not performed because a nerve is not suspected of being located at the site, a surgeon may cut out the nerve without recognizing it as a normal nerve. Therefore, the conventional nerve monitoring system has a limitation in that unpredicted nerve injury cannot be prevented.

In addition, a variety of kinds of surgical instruments are used in various steps of a surgical operation. By the way, the conventional nerve monitoring system entails a drawback in that a surgical instrument and a nerve probe are used separately from each other, and thus a user suffers from inconvenience of having to frequently replace the nerve probe in order to identify a relevant nerve during surgery.

In the meantime, an energy device for surgical operations has recently been developed and used as a surgical instrument for performing a rapid surgical operation while minimizing hemorrhage during surgery. Since such an energy device for surgical operations can perform hemostasis and detachment relatively rapidly compared to the conventional surgical instrument, it is widely used for purpose of reduction of surgery time and accurate hemostasis even in laparoscopic surgery, robotic surgery as well as general surgical operations. Examples of the energy device for surgical operations include an ultrasonic scalpel (representative product name: harmonic scalpel) using vibration of an ultrasonic wave of frequency 55,000 Hz, a bipolar vessel sealing system (representative product name: LigaSure) using a bipolar electric coagulator, and a thunderbeat having two functions, and the like.

However, a special attention must be paid to nerve injury in the use of the above-described energy device for surgical operations. Similarly to other surgical instruments, the energy device for surgical operations also has a structure in which it generates high heat at the periphery thererof along with the case where a surgeon causes an injury to nerves while cutting out body tissue, leading to the occurrence of nerve injury due to the high heat. It is reported that temporary nerve injury actually occurs frequently during the use of the energy device for surgical operations.

### DISCLOSURE OF INVENTION

### Technical Problem

Accordingly, the present invention has been made in order to solve the above-described problems occurring in the prior art, and it is an object of the present invention is to provide a novel type nerve probe attachable and detachable to and from a surgical instrument in which it includes a main body block and a connection cable, which are made of electrically conductive material based on a fact that many surgical instruments are made of metal and have an electrical conductivity, and by virtue of a structure in which the main body block includes a magnet body, a forceps or a hook so as to be detachably mounted to a general surgical instrument or includes a fitting groove formed therein so as to be detachably mounted to a protruding pin formed at a handle portion of the endoscopic surgical instrument or the robotic surgical instrument, an electric current is applied to body tissue by the surgical instrument made of an electrically conductive material so that real-time nerve identification and surgery to be performed simultaneously at body tissue coming into close contact with the surgical instrument only by use of the surgical instrument even without suffering from inconvenience of having to frequently replace a separate nerve probe and the surgical instrument, leading to promotion of rapid surgery and improvement of convenience of medical staffs.

Another object of the present invention is to provide a novel type nerve probe attachable and detachable to and from a surgical instrument in which a main body block includes a magnet or a forceps/a hook/a fitting groove so as to be attached to a rear end of the surgical instrument based on a fact that many surgical instruments are made of metal and have a property of being attracted to the magnet, so that the nerve probe can be used by being detachably attached to various forms of the surgical instruments (a general surgical instrument, an endoscopic surgical instrument, and a robotic surgical instrument) in a simple and easy manner, leading to promotion of acquisition of universality and enhancement of utility, as well as the nerve probe can be used easily without twisting of the wire irrespective of the kind and position of and any changes in posture of the surgical instrument, thus leading to optimization of an environment of surgery that is performed simultaneously with nerve identification.

Still another object of the present invention is to provide a novel type surgical energy device attached with a nerve probe in which the nerve probe is detachably mounted to a surgical instrument, and an electric wire for probe attached integrally to the surgical instrument is formed extendedly at a distal end of the surgical instrument so as to be electrically connected to the nerve probe so that nerve identification is performed by a nerve monitoring system along with the application of the current to body tissue coming into close contact with the surgical instrument by the electric wire for probe, and thus real-time nerve identification and surgery can be performed simultaneously at body tissue coming into close contact with the surgical instrument only by use of the surgical instrument, leading to promotion of rapid surgery and improvement of convenience of medical staffs.

In particular, yet another object of the present invention is to provide a novel type surgical energy device attached with a nerve probe in which it is applied to an ultrasonic scalpel, a bipolar vessel sealing system, or a thunderbeat that is an energy devices for surgical operations, which requires a special attention to be paid to nerve injury due to generation of heat so that nerve identification and early finding of nerve injury caused by heat can be performed in an accurate and smooth manner, leading to minimization of nerve injury during surgery by an energy device for surgical operations.

### Technical Solution

To achieve the above objects, in one aspect, the present invention provides a nerve probe 100 attachable and detachable to and from a surgical instrument, the nerve probe 100 including: a connection cable 120 connected to a power supply 300 and configured to transmit an electric current applied thereto from the power supply 300; and a main body block 110 formed as a block body of a predetermined size at a distal end of the connection cable 120 so as to be detachably attached to a surgical instrument 1 made of an electrically conductive material, the main body block 110 being made of an electrically conductive material so as to receive the electric current from the connection cable 120 for application to the surgical instrument 1, whereby the electric current is applied to body tissue with which the surgical instrument 1 comes into close contact during surgery through the connection cable 120, the main body block 110, and the surgical instrument 1, and an electric signal generated from a muscle movement by the applied electric current is detected through a signal-detecting electrode 230 attached to a preset point of a body region and then is inputted to a nerve monitoring system 200 to allow a nerve to be identified by electromyogram analysis.

In another aspect, the present invention provides a surgical energy device attached with a nerve probe, the surgical energy device including: a surgical instrument 1 used in a surgical operation; a nerve probe 100 detachably mounted to a preset portion of the surgical instrument 1; and an electric wire 140 for probe attached to the surgical instrument 1 so as to be electrically connected to the nerve probe 100, and disposed at a distal end of the surgical instrument 1, which comes into close contact with body tissue, to allow an electric current to be applied to body tissue, wherein the nerve probe 100 includes: a connection cable 120 configured to transmit the electric current applied thereto from the power supply 300; a main body block 110 formed as a block body of a predetermined size at a distal end of the connection cable 120 so as to be detachably attached to the surgical instrument 1, whereby the electric current transmitted from the power supply 300 through the connection cable 120 is applied to body tissue with which the surgical instrument 1 comes into close contact during surgery through the connection cable 120 of the nerve probe 100 and the electric wire 140 for probe of the surgical instrument 1, and an electric signal generated from a muscle movement by the electric current applied to the body tissue is detected through a signal-detecting electrode 230 attached to a preset point of a body region and then is inputted to a nerve monitoring system 200 to allow a nerve to be identified by electromyogram analysis.

### Advantageous Effects

The nerve probe attachable and detachable to and from a surgical instrument according to the present invention has an effect in that real-time nerve identification and surgery to be performed simultaneously at body tissue coming into close contact with the surgical instrument only by use of the surgical instrument even without suffering from inconvenience of having to frequently replace a separate nerve probe and the surgical instrument, leading to promotion of rapid surgery and improvement of convenience of medical staffs. In addition, the nerve probe attachable and detachable to and from a surgical instrument according to the present invention has an effect in that the main body block includes the magnet body or the forceps/hook/the fitting groove so as to be attached to the rear end of the surgical instrument so that the nerve probe can be used easily without twisting of the wire irrespective of the kind and position of and any changes in posture of the surgical instrument, thus leading to optimization of an environment of surgery that is performed simultaneously with nerve identification.

Further, the nerve probe attachable and detachable to and from a surgical instrument according to the present invention has an effect in that an electric wire for probe formed extendedly at a distal end of the surgical instrument to which the nerve probe is detachably mounted so that real-time nerve identification and surgery can be performed simultaneously at body tissue coming into close contact with the surgical instrument only by use of the surgical instrument, leading to promotion of rapid surgery and improvement of convenience of medical staffs.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is a diagrammatic view illustrating a conventional nerve probe provided and used separately from surgical instruments;

FIG. 2 is a diagrammatic view illustrating a configuration in which a nerve probe attachable and detachable to and from a surgical instrument according to an embodiment of the present invention is connected to a surgical instrument and a nerve monitoring system;

FIG. 3 is a cross-sectional view illustrating a nerve probe attachable and detachable to and from a surgical instrument according to an embodiment of the present invention, which includes a magnet body.

FIGS. 4 to 8 are views illustrating various modifications of a main body block constituting a nerve probe attachable and detachable to and from a surgical instrument according to an embodiment of the present invention;

FIG. 9 is a diagrammatic view illustrating a nerve probe attachable and detachable to and from a surgical instrument according to another embodiment of the present invention; diagrammatic view illustrating

FIG. 10 is a diagrammatic view illustrating a configuration in which a nerve probe attachable and detachable to and from a surgical instrument according to another embodiment of the present invention is attached to an endoscopic surgical instrument;

FIG. 11 is a diagrammatic view illustrating a configuration in which a nerve probe attachable and detachable to and from a surgical instrument according to another embodiment of the present invention is attached to a robotic surgical instrument;

FIG. 12 is a block diagram illustrating a configuration of a surgical energy device attached with a nerve probe according to an embodiment of the present invention;

FIG. 13 is a diagrammatic view illustrating a connection configuration between a surgical instrument and a nerve probe according to an embodiment of the present invention;

FIG. 14 is a diagrammatic view illustrating an arrangement configuration of an electric wire for probe in an ultrasonic scalpel to which a surgical energy device attached with a nerve probe according to an embodiment of the present invention is applied;

FIG. 15 is a diagrammatic view illustrating an arrangement configuration of an electric wire for probe in an bipolar vessel sealing system to which a surgical energy device attached with a nerve probe according to an embodiment of the present invention is applied; and

FIG. 16 is a diagrammatic view illustrating a circuit breaker provided at a bipolar vessel sealing system to which a surgical energy device attached with a nerve probe according to an embodiment of the present invention is applied.

### BEST MODE FOR CARRYING OUT THE INVENTION

A nerve probe 100 attachable and detachable to and from a surgical instrument according to an embodiment of the present invention includes: a connection cable 120 connected to a power supply 300 and configured to transmit an electric current applied thereto from the power supply 300; and a main body block 110 formed as a block body of a predetermined size at a distal end of the connection cable 120 so as to be detachably attached to a surgical instrument 1 made of an electrically conductive material, the main body block 110 being made of an electrically conductive material so as to receive the electric current from the connection cable 120 for application to the surgical instrument 1, whereby the electric current is applied to body tissue with which the surgical instrument 1 comes into close contact during surgery through the connection cable 120, the main body block 110, and the surgical instrument 1, and an electric signal generated from a muscle movement by the applied electric current is detected through a signal-detecting electrode 230 attached to a preset point of a body region and then is inputted to a nerve monitoring system 200 to allow a nerve to be identified by electromyogram analysis.

Herein, the main body block 110 may include a magnet body 130 so as to be detachably attached to the surgical instrument 1. In addition, the main body block 110 may include a forceps 112 consisting of a pair of clampers 1121a and 1121b so as to be detachably attached to the surgical instrument 1, and may include a claw-shaped hook 111 so as to be detachably attached to the surgical instrument 1. Alternatively, the main body block 110 may include a surgical instrument-connecting terminal 116 formed at a front end thereof, and the surgical instrument-connecting terminal 116 has a fitting groove 1161 formed therein so that the surgical instrument-connecting terminal 116 is detachably attached to a protruding pin 2 formed at a preset point of a rear end of each of an endoscopic surgical instrument 1′ and a robotic surgical instrument 1″.

In addition, the connection cable 120 includes: a conducting wire 121 along which an electric current is transmitted; and a cladding 122 configured to encircle the conducting wire 121 to insulate the conducting wire 121 from the outside, and arranged in expanded tubular form at a distal end thereof. The main body block 110 includes a main body 110a that is opened at a rear end thereof so that the opened rear end of the main body 110a constituting the main body block 110 can be inserted into the expanded distal end of the cladding 122 constituting the connection cable 120 to allow the conducting wire 121 and the main body 110a to be electrically connected to each other.

In addition, the main body block 110 includes: a cylindrical main body 110a made of an electrically conductive material, having a male thread machined face 114 formed on an outer circumferential surface thereof, and connected to the connection cable 120; and an auxiliary cap 110b made of an electrically conductive material, having a female thread machined face 115 formed on an inner circumferential surface of a rear end thereof so as to be coupled with the main body 110a, and including any one of a forceps 112 consisting of a pair of clampers 1121a and 1121b and a claw-shaped hook 111, which is formed at a front end thereof, so as to be retainingly secured to the surgical instrument 1. The forceps 112 that can be formed at the auxiliary cap 110b consists of a pair of hooked clampers which are pin-engaged with each other, and elasticity can be imparted in a direction in which the clampers 1121a and 1121b are engaged with each other by means of a torsion spring arranged at a pin-engagement portion of the forceps.

A surgical energy device attached with a nerve probe according to an embodiment of the present invention includes: a surgical instrument 1 used in a surgical operation; a nerve probe 100 detachably mounted to a preset portion of the surgical instrument 1; and an electric wire 140 for probe attached to the surgical instrument 1 so as to be electrically connected to the nerve probe 100, and disposed at a distal end of the surgical instrument 1, which comes into close contact with body tissue, to allow an electric current to be applied to body tissue, wherein the nerve probe 100 includes: a connection cable 120 configured to transmit the electric current applied thereto from the power supply 300; a main body block 110 formed as a block body of a predetermined size at a distal end of the connection cable 120 so as to be detachably attached to the surgical instrument 1, whereby the electric current transmitted from the power supply 300 through the connection cable 120 is applied to body tissue with which the surgical instrument 1 comes into close contact during surgery through the connection cable 120 of the nerve probe 100 and the electric wire 140 for probe of the surgical instrument 1, and an electric signal generated from a muscle movement by the electric current applied to the body tissue is detected through a signal-detecting electrode 230 attached to a preset point of a body region and then is inputted to a nerve monitoring system 200 to allow a nerve to be identified by electromyogram analysis.

The surgical instrument 1 may be an energy device for surgical operations, which generates heat to perform hemostasis. In particular, the surgical instrument 1 may be any one selected from the group consisting of an ultrasonic scalpel using ultrasonic energy, a bipolar vessel sealing system using bipolar high frequency energy, and a thunderbeat that is a dual energy device using both ultrasonic energy and bipolar high frequency energy. In addition, the surgical instrument 1 may be any one of an endoscopic surgical instrument and a robotic surgical instrument.

In the meantime, the surgical instrument 1 includes a protruding pin 2 formed protrudingly at a preset point of a rear end thereof, and the nerve probe 100 includes a surgical instrument-connecting terminal 116 formed at a front end thereof, the surgical instrument-connecting terminal 116 having a fitting groove 1161 formed therein so that the nerve probe 100 can be detachably mounted to the surgical instrument 1.

Further, the electric wire 140 for probe may be attached to a blade formed at a front end of the surgical instrument 1 so as to surround left and right lateral sides and a front side of the blade. Alternatively, the electric wire 140 for probe may be attached to at least one of a top side and a bottom side of the blade formed at the front end of the surgical instrument 1.

In the surgical energy device attached with a nerve probe according to an embodiment of the present invention, the electric wire 140 for probe is installed at a blade made of an electrically conductive material and configured to be applied with the electric current, and the surgical energy device may further include a circuit breaker 150 fixedly mounted at a preset point of the surgical instrument 1 so as to be electrically connected to the nerve probe 100 and the electric wire 140 for probe and configured to interrupt the electrical connection between nerve probe 100 and the electric wire 140 for probe upon the application of the electric current to the blade to prevent reverse flow of the current.

### BEST MODE

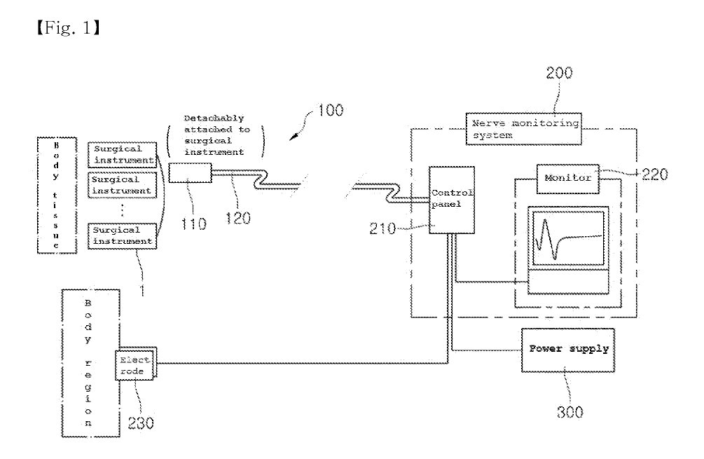

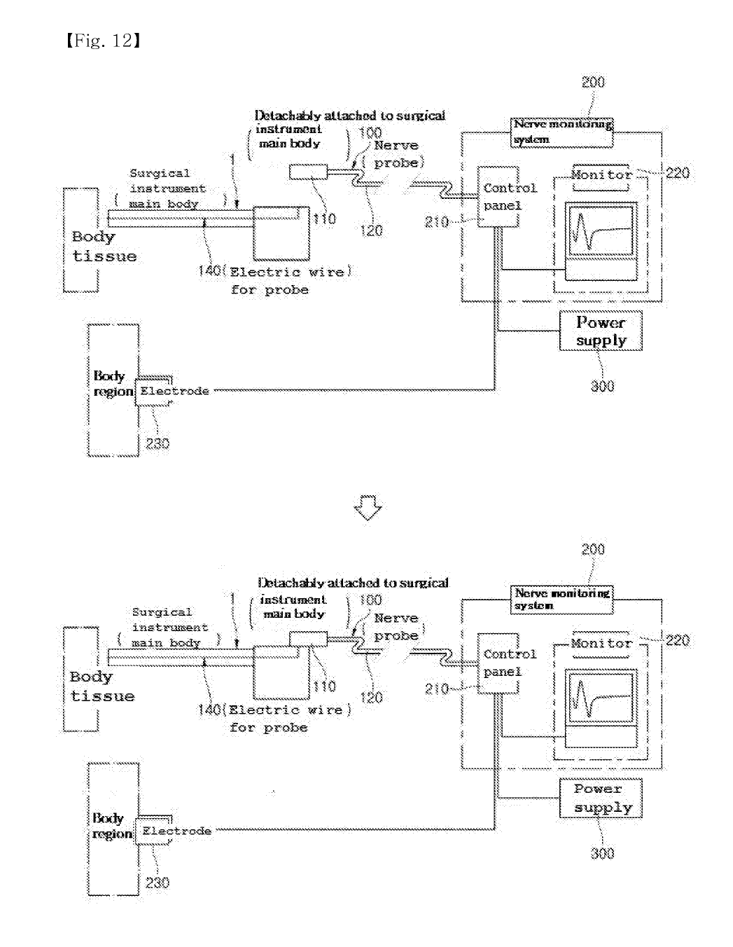

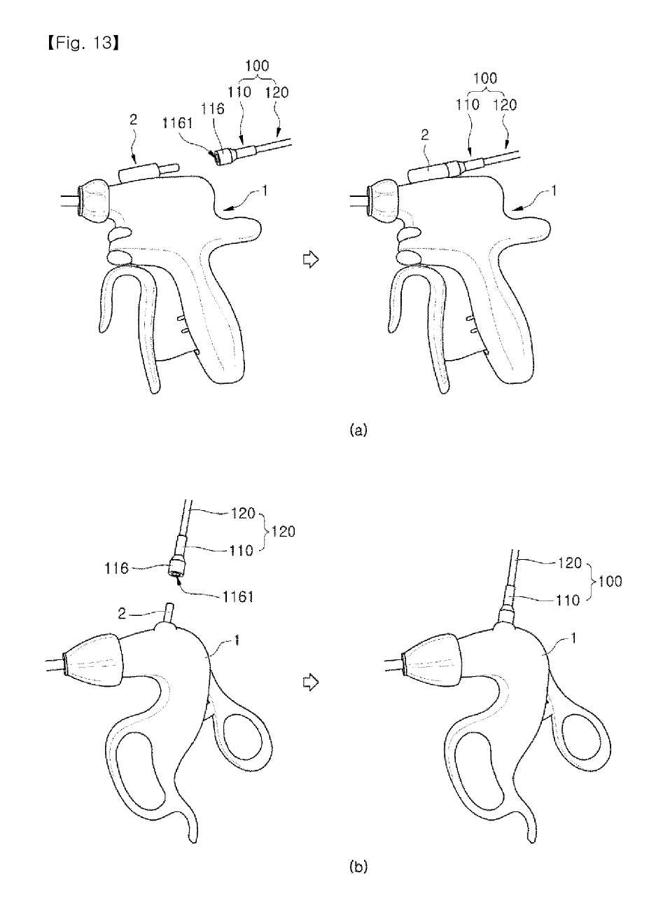

Hereinafter, preferred embodiments of the present invention will be described in further detail with reference to FIGS. 1 to 16 in the accompanying drawings. In the meantime, in the detailed description and the accompanying drawings, illustration and explanation on the construction and operation which a person skilled in the art can easily understand from a general surgical instrument, a general surgical instrument, endoscope, a robotic surgery, an endoscopic surgical instrument, a robotic surgical instrument, a nerve probe, an electric wire, a nerve monitoring system, a torsion spring, an energy device for surgical operations, an ultrasonic scalpel, a bipolar vessel sealing system, a thunderbeat, and the like will be briefly made or will be omitted to avoid redundancy. In particular, in the detailed description and the accompanying drawings, illustration and explanation on the detailed technical construction and operation of elements, which have no direct connection with the technical features of the present invention, will be omitted, and only the technical constructions directly related with the present invention will be briefly illustrated and explained.

The nerve probe 100 attachable and detachable to and from a surgical instrument according to an embodiment of the present invention includes a connection cable 120 and a main body block 110. As shown in FIG. 1, the electric current is applied to body tissue with which the surgical instrument 1 comes into close contact during surgery through the connection cable 120 connected to a control panel 210 to which a power supply 300 is connected so as to be supplied with electric power, the main body block 110, and the surgical instrument 1, and an electric signal generated from a muscle movement by the applied electric current is detected through a signal-detecting electrode 230 attached to a preset point of a body region and then is inputted to a nerve monitoring system 200 to allow a nerve to be identified by electromyogram analysis.

The connection cable 120 is connected to the power supply 300 and transmits an electric current applied thereto from the power supply 300 to the main body block 110.

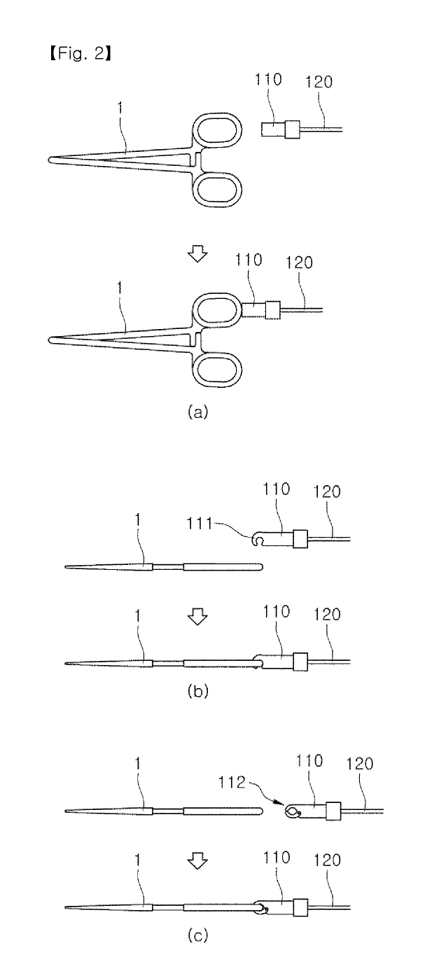

The main body block 110 is formed as a block body of a predetermined size at a distal end of the connection cable 120 so as to be detachably attached to the surgical instrument 1 made of an electrically conductive material. The main body block 110 is made of an electrically conductive material so as to receive the electric current from the connection cable 120 for application to the surgical instrument 1. Herein, the main body block 110 can include a magnet body 13 so as to be detachably attached to the surgical instrument 1 as shown in FIGS. 2(a) and 3. To this end, the main body block 110 has an electric conductivity similar to that of an electric wire and can be formed as a cylindrical body made of an iron material that is easily attracted to a magnet

The magnet body 130 can be disposed within the main body block 110. In particular, the main body block 110 according to an embodiment of the present invention includes a plurality of magnets arranged therein so as to be connected in series to each other to increase the magnitude of a magnetic force and allow a magnetic force of more than a certain magnitude to be uniformly exerted in multiple directions. By virtue of this, an attachment force of the main body block 110 to the surgical instrument 1 can be enhanced.

Alternatively, the main body block 110 may include a claw-shaped hook 111 so as to be detachably attached to the surgical instrument 1 as shown in FIG. 2(b), and may include a forceps 112 consisting of a pair of clampers 1121a and 1121b so as to be detachably attached to the surgical instrument 1 as shown in FIG. 2(c).

In the meantime, the connection cable 120 according to an embodiment of the present invention includes a conducting wire 121 and a cladding 122 as shown in FIG. 3.

The conducting wire 121 allows an electric current to flow therealong, and the cladding 122 encircle the conducting wire 121 to insulate the conducting wire 121 from the outside. Herein, the cladding 122 is arranged in expanded tubular form at a distal end thereof so that the main body block 110 can be inserted into the expanded distal end of the cladding 122.

The main body block 110 according to an embodiment of the present invention includes a main body 110a that is opened at a rear end thereof so that the opened rear end of the main body 110a constituting the main body block 110 can be inserted into the expanded distal end of the cladding 122 constituting the connection cable 120 to allow the conducting wire 121 and the main body 110a to be electrically connected to each other.

The nerve probe 100 attachable and detachable to and from a surgical instrument according to an embodiment of the present invention is used to be detachably attached to various kinds and sizes of surgical instrument 1.

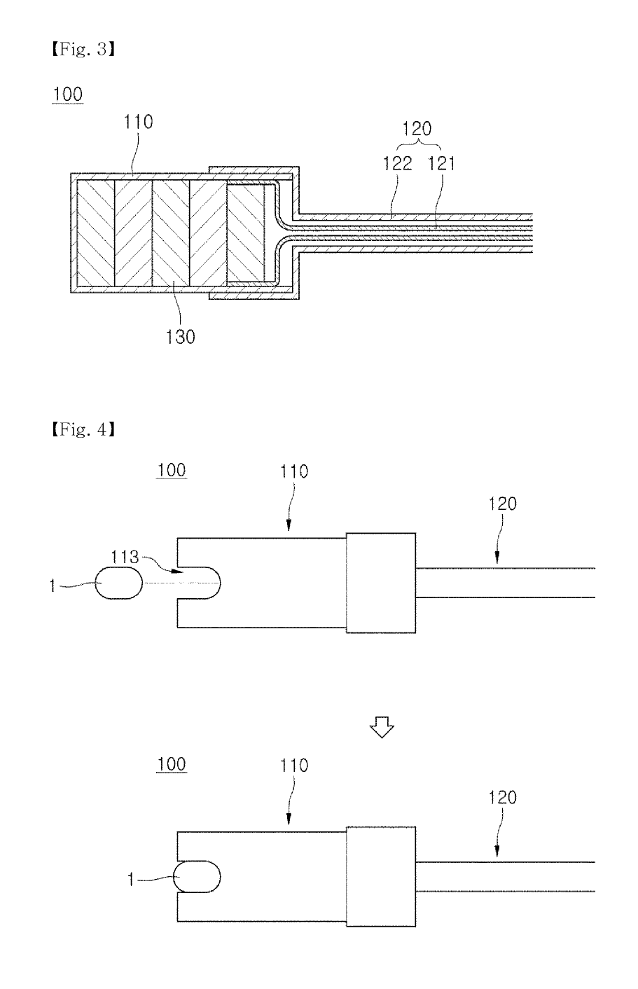

In the meantime, the nerve probe 100 attachable and detachable to and from a surgical instrument according to an embodiment of the present invention may include a main body block 110 of a structure in which the detachable fixation of the nerve probe 100 to the surgical instrument 1 is performed in a stable and smooth manner as show in FIGS. 4 to 8.

In other words, the main body block 110 includes a surgical instrument fitting groove 113 recessedly formed at a front end thereof to allow the surgical instrument 1 to be securely fitted into the surgical instrument fitting groove 113 of the main body block 110 as shown in FIG. 4 so that the engagement between the surgical instrument 1 and the nerve probe 100 can be held firmly and stably. As shown in FIG. 4, the main body block 110 includes a magnet body 130 disposed therein.

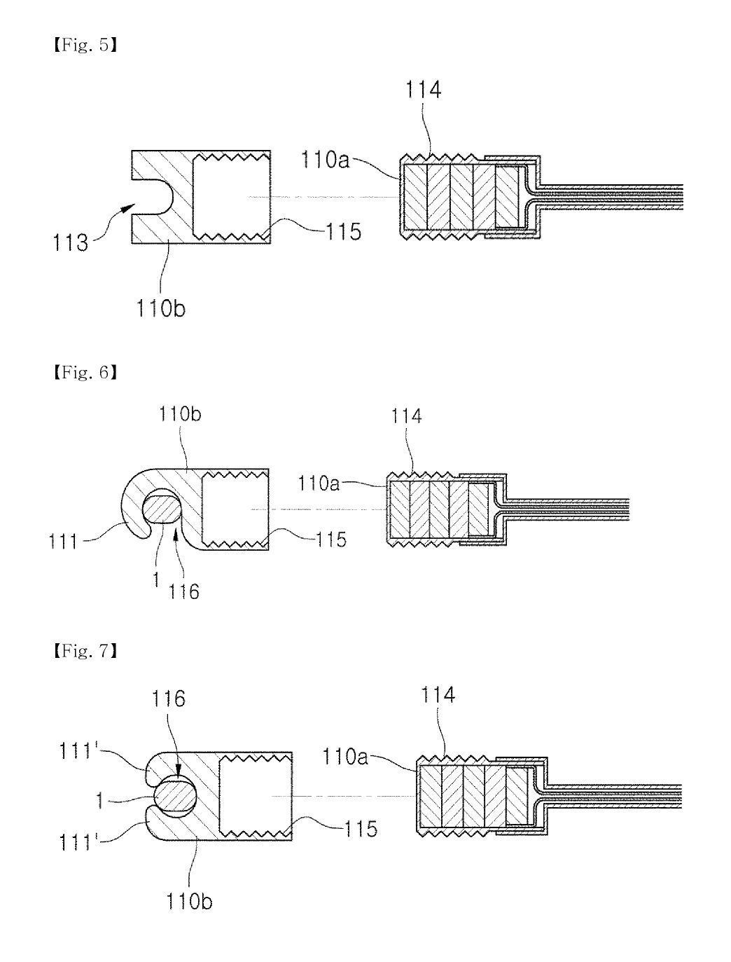

In addition, the main body block 110 includes a main body (110a) [symbol] auxiliary cap 110b, which are made of an electrically conductive material as shown in FIGS. 5 to 8.

The main body 110a is formed in a cylindrical shape, and has a male thread machined face 114 formed on an outer circumferential surface thereof, and is connected to the connection cable 120.

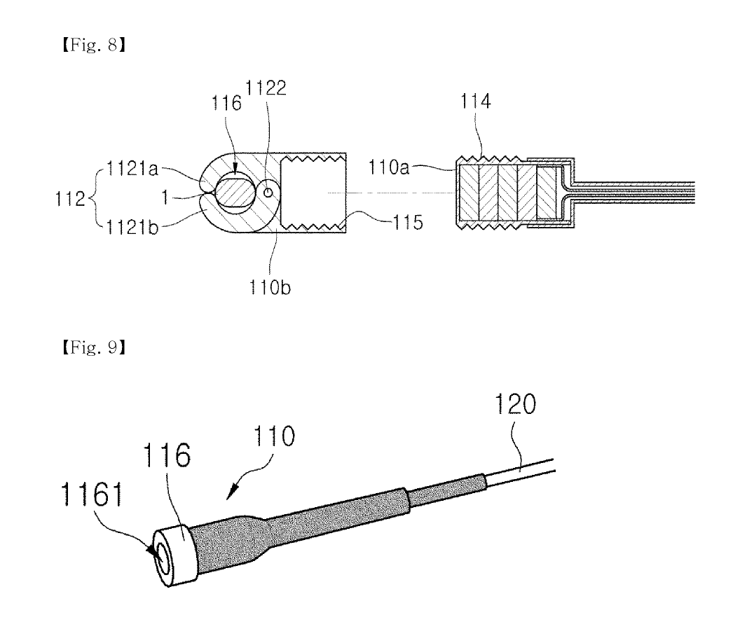

The auxiliary cap 110b has a female thread machined face 115 formed on an inner circumferential surface of a rear end thereof so as to be coupled with the main body 110a and retainingly secured to the surgical instrument 1. In order for the auxiliary cap 110b to be retainingly secured to the surgical instrument 1, the auxiliary cap 110b can include a surgical instrument fitting groove 113 formed at a front end thereof as shown in FIG. 5, a claw-shaped hook 111 formed at the front end thereof to have a surgical instrument fitting groove 116 formed therein as shown in FIG. 5, a pair of hook-shaped protrusions 111′ formed at the front end thereof to have a surgical instrument fitting groove 116 defined therebetween as shown in FIG. 7, and a forceps 112 consisting of a pair of clampers 1121a and 1121b formed at the front end thereof to have a surgical instrument fitting groove 116 defined therebetween as shown in FIG. 8. The auxiliary caps 110b show in FIGS. 5 to 8 may constitute a single set. As such, among various kinds of auxiliary caps 110b constituting the single set, an optimal auxiliary cap 110b conforming to the kind and size of the surgical instrument 1 can be selected and used. The main body 110a and the auxiliary cap 110b of the main body block 110 are configured so as to be detachably coupled to each other by means of a bolt-nut coupling structure, and thus various kinds of auxiliary caps 110b can be selectively mounted to the main body 110a.

Herein, the forceps 112 that can be formed at the auxiliary cap 110b consists of a pair of hooked clampers 1121a and 1121b which are pin-engaged with each other as shown in FIG. 8, and elasticity can be imparted in a direction in which the clampers 1121a and 1121b are engaged with each other by means of a torsion spring (not shown) arranged at a pin-engagement portion of the forceps. By virtue of this configuration, the nerve probe 100 of the present invention can be firmly secured to the surgical instrument 1.

The nerve probe 100 attachable and detachable to and from a surgical instrument according to an embodiment of the present invention as constructed above includes the main body block 110 and the connection cable 120.

By virtue of a structure in which the main body block 110 includes the magnet body 130, the forceps 112 or the hook 111 so as to be detachably mounted to the surgical instrument 1, an electric current is applied to body tissue by the surgical instrument made of an electrically conductive material so that nerve identification can be performed in real-time at body tissue coming into close contact with the surgical instrument only by use of the surgical instrument even without suffering from inconvenience of having to frequently replace a separate nerve probe and the surgical instrument. The use of nerve probe 100 attachable and detachable to and from a surgical instrument according to an embodiment of the present invention enables real-time nerve identification and surgery to be performed simultaneously, leading to promotion of rapid surgery and improvement of convenience of medical staffs.

In addition, in the nerve probe 100 attachable and detachable to and from a surgical instrument according to an embodiment of the present invention, the main body block 110 including the magnet body 130, the forceps 112, or the hook 111 is attached to the rear end of the surgical instrument 1, and thus the nerve probe 100 can be used by being detachably attached to various forms of the surgical instruments 1 in a simple and easy manner, leading to promotion of acquisition of universality and enhancement of utility, and the nerve probe can be used easily without twisting of the wire irrespective of the kind and position of and any changes in posture of the surgical instrument, thus leading to optimization of an environment of surgery that is performed simultaneously with nerve identification.

In the meantime, the nerve probe 100 attachable and detachable to and from a surgical instrument according to another embodiment of the present invention as shown in FIGS. 9 to 11 can be applied to a endoscopic surgical instrument 1′ and a robotic surgical instrument 1″.

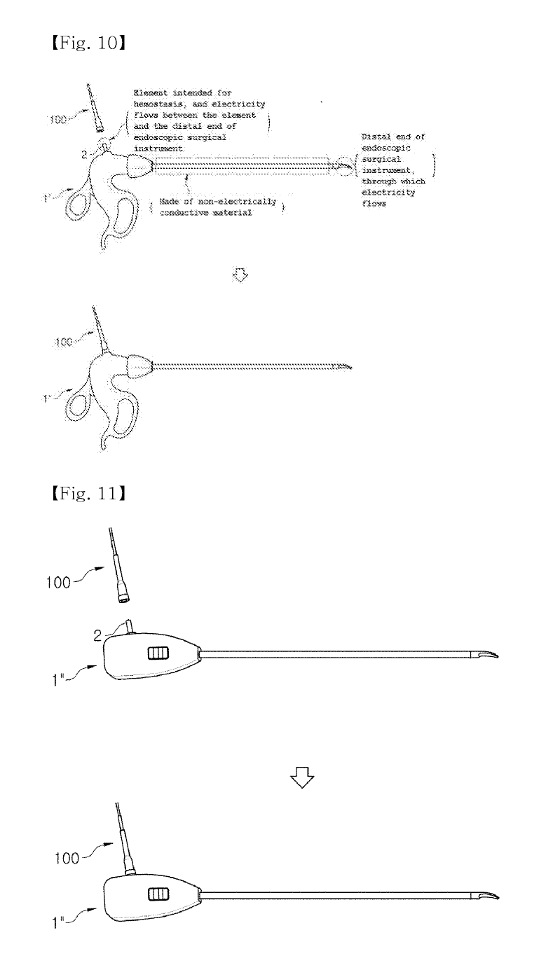

The endoscopic surgical instrument 1′ or the robotic surgical instrument 1″ allows a surgery to be performed through a small body cavity, and is made of a non-electrically conductive material. For this reason, it is impossible that the nerve probe 100 attachable and detachable to and from a surgical instrument according to an embodiment of the present invention including the magnet body or the hook/forceps is attached to the surface of the endoscopic surgical instrument 1′ or the robotic surgical instrument 1″ to cause electricity to flow therethrough. In the meantime, the endoscopic surgical instrument 1′ or the robotic surgical instrument 1″ is a device that allows a surgery to be performed through a small body cavity, and thus when hemorrhage occurs during surgery, a surgeon's hands do not reach a relevant surgical site. Thus, the protruding pin 2 for electrocautery is formed at the rear end of the endoscopic surgical instrument 1′ or the robotic surgical instrument 1″ so as to perform hemostasis. Other portions of the endoscopic surgical instrument 1′ or the robotic surgical instrument 1″ does not permit the flow of electricity, but the distal end portion of the endoscopic surgical instrument 1′ or the robotic surgical instrument 1″ and the protruding pin 2 attached with the electrocautery unit permit the flow of electricity. Therefore, the main body block 110 of the nerve probe 100 attachable and detachable to and from a surgical instrument according to another embodiment of the present invention includes a surgical instrument-connecting terminal 116 formed at the front end thereof and having a fitting groove 1161 formed therein as shown in FIG. 9 so that the nerve probe 100 can be detachably attached to the protruding pin 2 formed at a preset point of the rear end of the endoscopic surgical instrument 1′ or the robotic surgical instrument 1″ as shown in FIGS. 10 and 11.

Herein, the surgical instrument-connecting terminal 116 is made of an electrically conductive material and the outer surface of the main body block 110 can be coated with a non-electrically conductive material.

By virtue of this configuration, the presence and absence of a nerve can be identified by electromyogram through nerve stimulation according to the application of the current even during the use of the endoscopic surgical instrument 1′ or the robotic surgical instrument 1″.

In the nerve probe 100 attachable and detachable to and from a surgical instrument according to another embodiment of the present invention as constructed above, by virtue of a structure in which the main body block 110 includes the fitting groove 1161 formed therein so as to be detachably mounted to the endoscopic surgical instrument 1′ or the robotic surgical instrument 1″, an electric current is applied to body tissue by the endoscopic surgical instrument 1′ or the robotic surgical instrument 1″ so that nerve identification can be performed in real-time at body tissue coming into close contact with the endoscopic surgical instrument 1′ or the robotic surgical instrument 1″ only by use of the endoscopic surgical instrument 1′ or the robotic surgical instrument 1″ even without suffering from inconvenience of having to frequently replace a separate nerve probe and the surgical instrument. The use of nerve probe 100 attachable and detachable to and from a surgical instrument according to another embodiment of the present invention enables real-time nerve identification and surgery to be performed simultaneously, leading to promotion of rapid surgery and improvement of convenience of medical staffs.

Further, In the nerve probe 100 attachable and detachable to and from a surgical instrument according to another embodiment of the present invention as constructed above, the main body block 110 including the fitting groove 116 is attached to the protruding pin 2 formed the rear end of the endoscopic surgical instrument 1′ or the robotic surgical instrument 1″ so that the nerve probe 100 can be used by being detachably attached to the endoscopic surgical instrument 1′ or the robotic surgical instrument 1″ in a simple and easy manner, leading to promotion of acquisition of universality and enhancement of utility, and the nerve probe can be used easily without twisting of the wire irrespective of the kind and position of and any changes in posture of the endoscopic surgical instrument 1′ or the robotic surgical instrument 1″, thus leading to optimization of an environment of surgery that is performed simultaneously with nerve identification.

In the meantime, the surgical energy device 100 attached with a nerve probe according to an embodiment of the present invention includes a surgical instrument 1, a nerve probe 100, electric wire 140 for probe as shown in FIG. 6. The electric current transmitted from the power supply 300 through the connection cable 120 is applied to body tissue with which the surgical instrument 1 comes into close contact during surgery through the connection cable 120 of the nerve probe 100 and the electric wire 140 for probe of the surgical instrument 1, and an electric signal generated from a muscle movement by the electric current applied to the body tissue is detected through a signal-detecting electrode 230 attached to a preset point of a body region and then is inputted to a nerve monitoring system 200 to allow a nerve to be identified by electromyogram analysis. Herein, the electric signal refers to an electric signal transmitted from body tissue which comes into close contact with the surgical instrument 1 and the electric wire 140 for probe.

The surgical instrument 1 is an medical instrument used by medical staffs during surgical operations. The electric wire 140 for probe is attached integrally to the surgical instrument 1 and the nerve probe 100 is detachably mounted to the surgical instrument 1. In particular, an energy device for surgical operations, which generates heat to perform hemostasis, is used as the surgical instrument 1 according to an embodiment of the present invention. Of course, the surgical instrument 1 is not limited thereto.

As the energy device for surgical operations for implementing the surgical instrument 1, an ultrasonic scalpel using ultrasonic energy, a bipolar vessel sealing system using bipolar high frequency energy, and a thunderbeat that is a dual energy device using both ultrasonic energy and bipolar high frequency energy can be used. In addition, the energy device for surgical operations for implementing the surgical instrument 1 may be either an endoscopic surgical instrument or a robotic surgical instrument.

Herein, the surgical instrument 1 according to an embodiment of the present invention includes a protruding pin 2 formed protrudingly at a preset point of a rear end thereof. In the case where the surgical instrument 1 is an endoscopic surgical instrument or a robotic surgical instrument, the protruding pin 2 is attached with an electrocautery unit. In the surgical instrument 1 according to an embodiment of the present invention, the nerve probe 100 is mounted to the protruding pin 2.

The nerve probe 100 is detachably mounted to a preset portion of the surgical instrument 1. The nerve probe 100 according to an embodiment of the present invention includes a surgical instrument-connecting terminal 116 formed at a front end thereof, the surgical instrument-connecting terminal 116 having a fitting groove 1161 formed therein so that the nerve probe 100 is detachably mounted to the surgical instrument 1. In addition, the nerve probe 100 according to an embodiment of the present invention includes a connection cable 120 and a main body block 110.

The connection cable 120 transmits an electric current applied thereto from the power supply 300.

The main body block 110 is formed as a block body of a predetermined size at a distal end of the connection cable 120 so as to be detachably attached to the surgical instrument 1. The surgical instrument-connecting terminal 116 having a fitting groove 1161 formed therein is formed at a front end of the main body block 110.

The electric wire 140 for probe is attached integrally to the surgical instrument 1 and is electrically connected to the nerve probe 100. Herein, the electric wire 140 for probe may be formed exposedly on an outer surface of the surgical instrument 1, and may be arranged insertedly within the surgical instrument 1. The exposed formation or the inserted arrangement of the electric wire 40 is determined depending on the shape and structure of the surgical instrument 1.

The electric wire 140 for probe is disposed at a distal end of the surgical instrument 1, which comes into close contact with body tissue, to allow the electric current to be applied to body tissue. The electric wire 140 for probe preferably is attached to a blade formed at a front end of the surgical instrument 1 so as to surround left and right lateral sides and a front side of the blade. Alternatively, the electric wire 140 for probe may be attached to at least one of a top side and a bottom side of the blade formed at the front end of the surgical instrument 1.

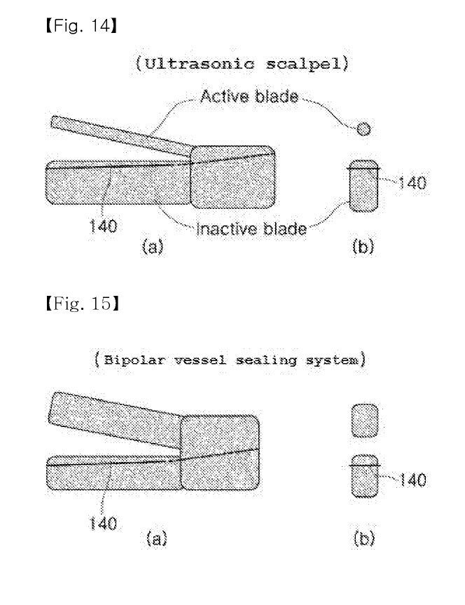

Herein, the ultrasonic scalpel for implementing the surgical instrument 1 includes an upper blade acting as an “active blade” that is vibrated by an ultrasonic wave of frequency 55,000 Hz to generate heat, and a lower blade acting as an “inactive blade” that is inactivated to function to protect body tissue. As shown in FIG. 14, the electric wire 140 for probe of the surgical instrument 1 implemented as the ultrasonic scalpel is attached to the lower blade so as to surround left and right lateral sides and a front side of the lower blade as the “inactive blade”. Specifically, the electric wire 140 for probe is preferably attached to the lower blade so as to be positioned at an adjacent position within a spaced height ranging from 0.1 to 10 mm from a contact face where the upper and lower blades are in close contact with each other. Of course, the attachment position of the electric wire 140 for probe is not limited thereto.

Moreover, the bipolar vessel sealing system for implementing the surgical instrument 1 is a medical instrument that cuts out a blood vessel while performing hemostasis of the blood vessel by bipolar electrocautery. Thus, electricity flows through the upper and lower blades. As shown in FIG. 15, the electric wire 140 for probe of the surgical instrument 1 implemented as the bipolar vessel sealing system is preferably attached to the lower blade so as to be positioned at an adjacent position within a spaced height ranging from 0.1 to 10 mm from a contact face where the upper and lower blades are in close contact with each other. Of course, the attachment position of the electric wire 140 for probe is not limited thereto.

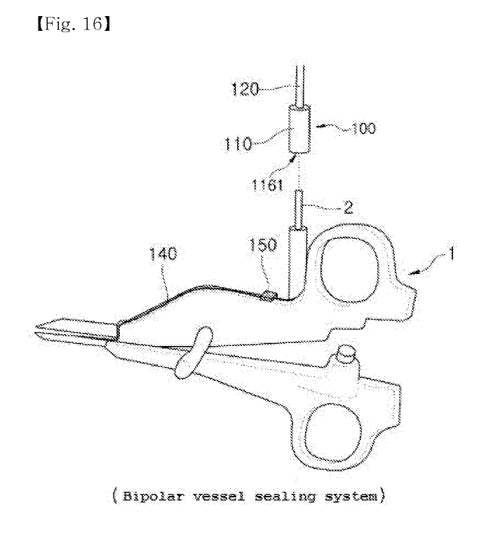

As in the bipolar vessel sealing system, in the case where the electric wire 140 for probe is installed at the blade made of an electrically conductive material so as to be applied with the electric current, when the electric current is applied to the surgical instrument 1 in order for the surgical instrument 1 to perform its own intrinsic function, the current applied to the blade may reversely flow through the electric wire 140 for probe. In order to prevent this reverse flow of the current, a circuit breaker 150 can be installed at the surgical instrument 1 as shown in FIG. 16. The circuit breaker 150 is fixedly mounted at a preset point of the surgical instrument 1 so as to be electrically connected to the nerve probe 100 and the electric wire 140 for probe, and interrupts the electrical connection between nerve probe 100 and the electric wire 140 for probe upon the application of the electric current to the blade to prevent reverse flow of the current.

In the surgical energy device attached with a nerve probe according to an embodiment of the present invention as constructed above, the nerve probe 100 is detachably mounted to a surgical instrument 1, and an electric wire 140 for probe attached integrally to the surgical instrument 1 is formed extendedly at a distal end of the surgical instrument 1 so as to be electrically connected to the nerve probe 100 so that real-time nerve identification is performed by a nerve monitoring system 200 along with the application of the current to body tissue coming into close contact with the surgical instrument 1 by the electric wire for probe, and thus nerve identification and surgery can be performed simultaneously at body tissue coming into close contact with the surgical instrument only by use of the surgical instrument, leading to promotion of rapid surgery and improvement of convenience of medical staffs. In particular, the surgical energy device 1 attached with a nerve probe according to an embodiment of the present invention is applied to an ultrasonic scalpel, a bipolar vessel sealing system, or a thunderbeat that is an energy devices for surgical operations, which requires a special attention to be paid to nerve injury due to generation of heat so that nerve identification and early finding of nerve injury caused by heat can be performed in an accurate and smooth manner, leading to minimization of nerve injury during surgery by an energy device for surgical operations.

While the nerve probe attachable and detachable to and from a surgical instrument and the surgical energy device attached with a nerve probe according to the preferred embodiments of the present invention has been described and illustrated in connection with specific exemplary embodiments with reference to the accompanying drawings, it will be readily appreciated by those skilled in the art that it is merely illustrative of the preferred embodiments of the present invention and various modifications and changes can be made thereto within the technical spirit and scope of the present invention.

### INDUSTRIAL APPLICABILITY

The nerve probe attachable and detachable to and from a surgical instrument according to the present invention can be used by being detachably attached to various forms of the surgical instruments (a general surgical instrument, an endoscopic surgical instrument, and a robotic surgical instrument) in a simple and easy manner, leading to promotion of acquisition of universality and enhancement of utility. In addition, the surgical energy device attached with a nerve probe according to the present invention is effectively applied to the energy devices for surgical operations, which requires a special attention to be paid to nerve injury due to generation of heat so that nerve identification and early finding of nerve injury caused by heat can be performed in an accurate and smooth manner, leading to stability of the energy device for surgical operations and enhancement of utility.

1. A nerve probe attachable and detachable to and from a surgical instrument, comprising:
a connection cable 120 connected to a power supply 300 and configured to transmit an electric current applied thereto from the power supply 300; and
a main body block 110 formed as a block body of a predetermined size at a distal end of the connection cable 120 so as to be detachably attached to a surgical instrument 1 made of an electrically conductive material, the main body block 110 being made of an electrically conductive material so as to receive the electric current from the connection cable 120 for application to the surgical instrument 1,
whereby the electric current is applied to body tissue with which the surgical instrument 1 comes into close contact during surgery through the connection cable 120, the main body block 110, and the surgical instrument 1, and an electric signal generated from a muscle movement by the applied electric current is detected through a signal-detecting electrode 230 attached to a preset point of a body region and then is inputted to a nerve monitoring system 200 to allow a nerve to be identified by electromyogram analysis.

2. The nerve probe according to claim 1, wherein the main body block 110 comprises a magnet body 13 so as to be detachably attached to the surgical instrument 1.

3. The nerve probe according to claim 2, wherein the main body block 110 comprises any one of a forceps 112 consisting of a pair of clampers 1121a and 1121b, and a claw-shaped hook 111 so as to be detachably attached to the surgical instrument 1.

4. The nerve probe according to claim 1, wherein the main body block 110 comprises a surgical instrument-connecting terminal 116 formed at a front end thereof, and the surgical instrument-connecting terminal 116 has a fitting groove 1161 formed therein so that the surgical instrument-connecting terminal 116 is detachably attached to a protruding pin 2 formed at a preset point of a rear end of each of an endoscopic surgical instrument 1′ and a robotic surgical instrument 1″.

5. The nerve probe according to claim 1, wherein the main body block 110 comprises:
a cylindrical main body 110a made of an electrically conductive material, having a male thread machined face 114 formed on an outer circumferential surface thereof, and connected to the connection cable 120; and
an auxiliary cap 110b made of an electrically conductive material, having a female thread machined face 115 formed on an inner circumferential surface of a rear end thereof so as to be coupled with the main body 110a, and including any one of a forceps 112 consisting of a pair of clampers 1121a and 1121b and a claw-shaped hook 111, which is formed at a front end thereof, so as to be retainingly secured to the surgical instrument 1.

6. A surgical energy device attached with a nerve probe, comprising:
a surgical instrument 1 used in a surgical operation;
a nerve probe 100 detachably mounted to a preset portion of the surgical instrument 1; and
an electric wire 140 for probe attached to the surgical instrument 1 so as to be electrically connected to the nerve probe 100, and disposed at a distal end of the surgical instrument 1, which comes into close contact with body tissue, to allow an electric current to be applied to body tissue,
wherein the nerve probe 100 comprises:
a connection cable 120 configured to transmit the electric current applied thereto from the power supply 300;
a main body block 110 formed as a block body of a predetermined size at a distal end of the connection cable 120 so as to be detachably attached to the surgical instrument 1,
whereby the electric current transmitted from the power supply 300 through the connection cable 120 is applied to body tissue with which the surgical instrument 1 comes into close contact during surgery through the connection cable 120 of the nerve probe 100 and the electric wire 140 for probe of the surgical instrument 1, and an electric signal generated from a muscle movement by the electric current applied to the body tissue is detected through a signal-detecting electrode 230 attached to a preset point of a body region and then is inputted to a nerve monitoring system 200 to allow a nerve to be identified by electromyogram analysis.

7. The surgical energy device according to claim 6, wherein the surgical instrument 1 is implemented as an energy device for surgical operations, which generates heat to perform hemostasis, the surgical instrument 1 being any one selected from the group consisting of an ultrasonic scalpel using ultrasonic energy, a bipolar vessel sealing system using bipolar high frequency energy, and a thunderbeat that is a dual energy device using both ultrasonic energy and bipolar high frequency energy.

8. The surgical energy device according to claim 6, wherein the surgical instrument 1 is any one of an endoscopic surgical instrument and a robotic surgical instrument.

9. The surgical energy device according to claim 6, wherein the surgical instrument 1 comprises a protruding pin 2 formed protrudingly at a preset point of a rear end thereof, and the nerve probe 100 comprises a surgical instrument-connecting terminal 116 formed at a front end thereof, the surgical instrument-connecting terminal 116 having a fitting groove 1161 formed therein so that the nerve probe 100 is detachably mounted to the surgical instrument 1.

10. The surgical energy device according to claim 6, wherein the electric wire 140 for probe is attached to a blade formed at a front end of the surgical instrument 1 so as to surround left and right lateral sides and a front side of the blade.

11. The surgical energy device according to claim 6, wherein the electric wire 140 for probe is installed at a blade made of an electrically conductive material and configured to be applied with the electric current, and
wherein the surgical energy device further comprises a circuit breaker 150 fixedly mounted at a preset point of the surgical instrument 1 so as to be electrically connected to the nerve probe 100 and the electric wire 140 for probe, the circuit breaker being configured to interrupt the electrical connection between nerve probe 100 and the electric wire 140 for probe upon the application of the electric current to the blade to prevent reverse flow of the current.

12. The nerve probe according to claim 1, wherein the main body block 110 comprises any one of a forceps 112 consisting of a pair of clampers 1121a and 1121b, and a claw-shaped hook 111 so as to be detachably attached to the surgical instrument 1.
